(19) United States
(12) Patent Application Publication
(10) Pub. No.: US 2014/0088453 A1
(43) Pub. Date: Mar. 27, 2014

(54) Instrument Penetration Detector Using Dynamic Frequency Adjustment, and Method of Operation

(71) Applicant: SensorMed, Inc., Knoxville, TN (US)

(72) Inventors: Michael C. Doody, Knoxville, TN (US); William T. Milam, Maryville, TN (US); M. Christopher Doody, JR., Knoxville, TN (US)

(21) Appl. No.: 13/626,338

(22) Filed: Sep. 25, 2012

(51) Int. Cl. A61B 5/06; A61B 5/053; A61B 5/00; A61B 19/00

(52) U.S. Cl. CPC A61B 5/061; A61B 19/5244; A61B 5/0538; A61B 5/6848
USPC 600/547

(57) ABSTRACT

A method, and an apparatus to perform the method, of determining a location of a medical instrument in a patient during a medical procedure, the method including connecting at least a portion of the medical instrument to a first body region of the patient, propagating a plurality of signals at different frequencies along a conductive path of the medical instrument, measuring one or more feedback parameters corresponding to each of the plurality of signals at the first body region, determining an operational frequency from the different frequencies according to a comparison of the one or more feedback parameters, propagating a signal having the operational frequency along the conductive path as the medical instrument penetrates the body of the patient during the medical procedure, and measuring the one or more feedback parameters corresponding to the operational frequency to determine a penetration location of the medical instrument in the body of the patient.

### CROSS-REFERENCE TO RELATED APPLICATIONS

Not Applicable

### FIELD OF INVENTION

The present general inventive concept relates to the field of medical procedures, and, more particularly, to a system and apparatus to detect instrument penetration in a patient's body, and a method of detecting a location of the instrument penetration.

### BACKGROUND

Prior techniques for surgery requiring insertion of a needle or a small diameter probe into and through the anatomical regions of a patient include laparoscopic surgery with a laparoscope inserted into the interior of the abdominal cavity. Another surgical technique includes insertion of a verres needle through tissue layers and into the abdominal cavity at about the umbilical region as a part of an insufflation technique, which is the act of blowing a vapor, gas, and/or air into a body cavity such as the abdominal cavity for sufficient distension of the cavity to allow for examination and manipulation of the cavity contents. Still another instrument that can be used for the initial patient access is a trocar. Insertion techniques for injections of medications include insertion of a needle and/or a cannula/catheter through the skin and into blood vessels or other body cavities for injection of fluids. Conventional insertion techniques typically require the practitioner to be able to judge by the feel of the insertion of the instrument as to whether the instrument has reached a targeted body region, such as a vein, layer of tissue, or body cavity. For example, during investigations of the abdominal cavity, a practitioner determines the progress of insertion of the penetrating needle end through the tissue layers of the umbilical region of the abdominal cavity.

Some prior art techniques utilized by practitioners include detection of sound as the needle end penetrates, and/or the utilization of touch and feel of the physical resistance, or lack of resistance, against the needle end during penetration. An additional prior technique includes measuring changes in pressure maintained at the penetrating end of a verres needle during penetration of the multiple layers of the umbilical region of the abdomen. The multiple layers of the umbilical region include the outer skin layer, a fat cell layer of variable thickness, a fascia layer of variable tissue thickness and abdominal muscles, a peritoneum layer, and the abdominal cavity. Each of the layers of the umbilical region may vary in depth between patients, and there may be the presence of scar tissue, therefore the penetration of a needle or a similar probe during the insufflation technique requires an extremely delicate sequence of steps.

It is beneficial to medical practitioners to have a reliably reproducible monitoring system having feedback notification that indicates to an operator when each tissue layer is penetrated and when a body cavity is penetrated by an insertion end of a needle or probe. Further, it is beneficial to have a method for operation of a system utilized for monitoring the stages of penetration of an insertion end of a needle or probe through each one of a plurality of outer layers covering a body cavity of a patient.

U.S. Pat. No. 6,603,997 describes a probe penetration detector system that monitors feedback signals to detect changes that relate to the location of the probe. It has been discovered that a signal frequency selected for a medical procedure conducted on one patient may not be as effective when used for another patient, due to such various factors as the wide variety in human body sizes, densities, bio-impedance, and so on.

### BRIEF SUMMARY

The present general inventive concept provides a method, and a system and apparatus to perform the method, of instrument penetration detection that generates a signal that may be optimized according to various characteristics of different respective patients, so that detected feedback factors may more efficiently indicate a body location of a medical instrument, such as a probe, needle, etc.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by a method of determining a location of a medical instrument in a patient during a medical procedure, the method including connecting at least a portion of the medical instrument to a first body region of the patient, propagating a plurality of signals at different frequencies along a conductive path of the medical instrument, measuring one or more feedback parameters corresponding to each of the plurality of signals at the first body region, determining an operational frequency from the different frequencies according to a comparison of the one or more feedback parameters, propagating a signal having the operational frequency along the conductive path as the medical instrument penetrates the body of the patient during the medical procedure, and measuring the one or more feedback parameters corresponding to the operational frequency to determine a penetration location of the medical instrument in the body of the patient.

The method may further include generating at least one indicator to indicate the medical instrument has reached a targeted body region of the patient.

The connecting may include injecting the medical instrument into the body of the patient.

The medical instrument may be injected approximately one centimeter into the body of the patient.

The feedback parameters may include voltage standing wave ratio (VSWR), angle of reflective coefficient, reactance, impedance, phase shift coefficient, return power loss, reflected power, propagated power, reflection coefficient, resistance, capacitance, inductance, admittance, reflectance, absorbance, transmittance, transmission loss, time domain reflectometry, or any combination thereof.

The method may further include storing the detected one or more feedback parameters with information associating the detected one or more feedback parameters with the respective corresponding frequencies.

The medical instrument may be a probe, trocar, cannula, or needle.

The medical instrument may be at least partially covered with an insulating material, having at least a portion of a distal end of the medical instrument connected to the patient exposed to contact the patient.

The plurality of signals at different frequencies may be propagated successively.

The frequencies of the plurality of signals may be incremented by a constant value.

According to various examples, the frequencies of the plurality of signals may be incremented by 3, 5, or 10 MHz, although various other frequencies may be used.

According to various examples, a quantity of 5, 7, 10, or 15 of the signals at different frequencies may be propagated successively, although various other quantities may be used.

The plurality of signals at different frequencies may be propagated simultaneously in a broadband signal.

The measuring may include incrementally adjusting band pass receiving circuitry to selectively receive channels corresponding to the plurality of signals at different frequencies in the broadband signal.

The method may further include generating at least one indicator to indicate the signal having the operational frequency is being propagated along the conductive path.

The at least one indicator may include at least one audible indicator, at least one visual indicator, or any combination thereof.

At least one completion tone may be emitted in response to the signal having the operational frequency being propagated along the conductive path.

At least one processing tone may be emitted in response to the determining of the operational frequency being in process.

A completion visual indicator may be turned on in response to the signal having the operational frequency being propagated along the conductive path.

A processing visual indicator may be turned on in response to the determining of the operation frequency being in process.

The foregoing and/or other aspects and advantages of the present general inventive concept may also be achieved by a system to determine a location of a medical instrument in a patient during a medical procedure, the system including a signal generator to propagate a plurality of signals at different frequencies along a conductive path of the medical instrument, a measuring unit to measure one or more feedback parameters corresponding to each of the plurality of signals when the medical instrument is connected to a first body region of the patient, a comparing unit to compare the one or more feedback parameters of the respective signals, and a determining unit to determine an operational frequency from the different frequencies according to the comparison, wherein the signal generator propagates a signal having the operational frequency along the conductive path as the medical instrument penetrates the body of the patient during the medical procedure, and the measuring unit measures the one or more feedback parameters corresponding to the operational frequency to determine a penetration location of the medical instrument in the body of the patient.

The system may further include at least one indicator to indicate the medical instrument has reached a targeted body region of the patient.

The signal generator, measuring unit, comparing unit, and determining unit may be provided to the body of the medical instrument.

The feedback parameters may include voltage standing wave ratio (VSWR), angle of reflective coefficient, reactance, impedance, phase shift coefficient, return power loss, reflected power, propagated power, reflection coefficient, resistance, capacitance, inductance, admittance, reflectance, absorbance, transmittance, transmission loss, time domain reflectometry, or any combination thereof.

The system may further include a memory to store the detected one or more feedback parameters with information associating the detected one or more feedback parameters with the respective corresponding frequencies.

The medical instrument may be a probe, trocar, cannula, or needle.

The medical instrument may be at least partially covered with an insulating material, having at least a portion of a distal end of the medical instrument exposed to contact the patient.

The signal generator may successively propagate the plurality of signals at different frequencies.

The signal generator may increment the frequencies of the plurality of signals by a constant value.

The signal generator may increment the frequencies of the plurality of signals by 3, 5, or 10 MHz, although various other frequencies may be used.

The signal generator may successively propagate a quantity of 5, 7, 10, or 15 of the signals at different frequencies, although various other quantities may be used.

The signal generator may propagate the plurality of signals at different frequencies simultaneously in a broadband signal.

The measuring unit may include band pass receiving circuitry to selectively detect specific channels with an overall range of the broadband signal.

The system may further include at least one indicator to indicate the propagation along the conductive path of the signal having the operational frequency.

The at least one indicator may include at least one audible indicator, at least one visual indicator, or a combination thereof.

The at least one audible indicator may emit at least one completion tone in response to the propagation along the conductive path of the signal having the operational frequency.

The at least one audible indicator may emit at least one processing tone in response to the determining of the operational frequency being in process.

The at least one indicator may include a first visual indicator that is turned on in response to the propagation along the conductive path of the signal having the operational frequency.

The at least one indicator may include a second visual indicator that is turned on in response to the determining of the operational frequency being in process.

The first visual indicator may be green, and the second visual indicator may be red.

The foregoing and/or other aspects and advantages of the present general inventive concept may also be achieved by a processor readable storage medium having recorded thereon a program to cause a processor to perform a method of determining a location of a medical instrument in a patient during a medical procedure, the method including connecting at least a portion of the medical instrument to a first body region of the patient, propagating a plurality of signals at different frequencies along a conductive path of the medical instrument, measuring one or more feedback parameters corresponding to each of the plurality of signals at the first body region, determining an operational frequency from the different frequencies according to a comparison of the one or more feedback parameters, propagating a signal having the operational frequency along the conductive path as the medical instrument penetrates the body of the patient during the medical procedure, and measuring the one or more feedback parameters corresponding to the operational frequency to determine a penetration location of the medical instrument in the body of the patient.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIG. 1 illustrates a medical instrument penetration detector system according to an example embodiment of the present general inventive concept;

FIG. 2 illustrates a medical instrument penetration detector system according to another example embodiment of the present general inventive concept;

FIG. 3 illustrates a partial schematic view of various elements of the system illustrated in FIG. 1;

FIG. 4 illustrates a medical instrument penetration detector system according to another example embodiment of the present general inventive concept;

FIG. 5 illustrates a medical instrument penetration detector system according to yet another example embodiment of the present general inventive concept;

FIGS. 6A-6B illustrate a front and side view of a conductive medical instrument provided with signal generating electronics according to an example embodiment of the present general inventive concept;

FIG. 7 illustrates a medical procedure using the conductive medical instrument illustrated in FIG. 6;

FIG. 8 illustrates a flow chart of a method of dynamic frequency adjustment of a conductive medical instrument according to an example embodiment of the present general inventive concept;

FIG. 9 illustrates a timeline over which a plurality of generated signal frequencies are increasingly incremented according to an example embodiment of the present general inventive concept;

FIG. 10 illustrates a timeline over which a plurality of generated signal frequencies are simultaneously generated according to an example embodiment of the present general inventive concept; and

FIGS. 11A-11G are graphs illustrating changes in a feedback parameter as a conductive medical instrument is moved to various body locations.

### DETAILED DESCRIPTION

Reference will now be made to various example embodiments of the present general inventive concept, examples of which are illustrated in the accompanying drawings and illustrations. The example embodiments described herein are presented in order to explain the present general inventive concept by referring to the figures.

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The described progression of processing operations described are merely examples, however, and the sequence of operations is not limited to that set forth herein and may be changed as is known in the art, with the exception of operations necessarily occurring in a certain order. Also, description of well-known functions and constructions may be omitted for increased clarity and conciseness.

Various examples of the present general inventive concept, such as those described herein, may involve a variety of procedures such as surgical procedures or exams, as well as more relatively simple procedures such as drawing blood. Thus, these procedures may generally be referred to as medical procedures, and may involve any such procedure in which a medical device is introduced into a body, and may be equally applicable to both human and veterinary patients. Similarly, a variety of medical devices or instruments may be used to penetrate various body locations in these medical procedures. Examples of such instruments, some of which are described herein, may include a probe, cannula, trocar, needle, and so on. However, the several examples of medical procedures and instruments to which the present general inventive concept may be applied are not limited by the examples described herein. Also, it will be understood that the present general inventive concept is not limited to the components as illustrated in the various embodiments and illustrations, as various components may be omitted and/or added, may be combined into single components and/or modules, and/or may be provided as further separated components.

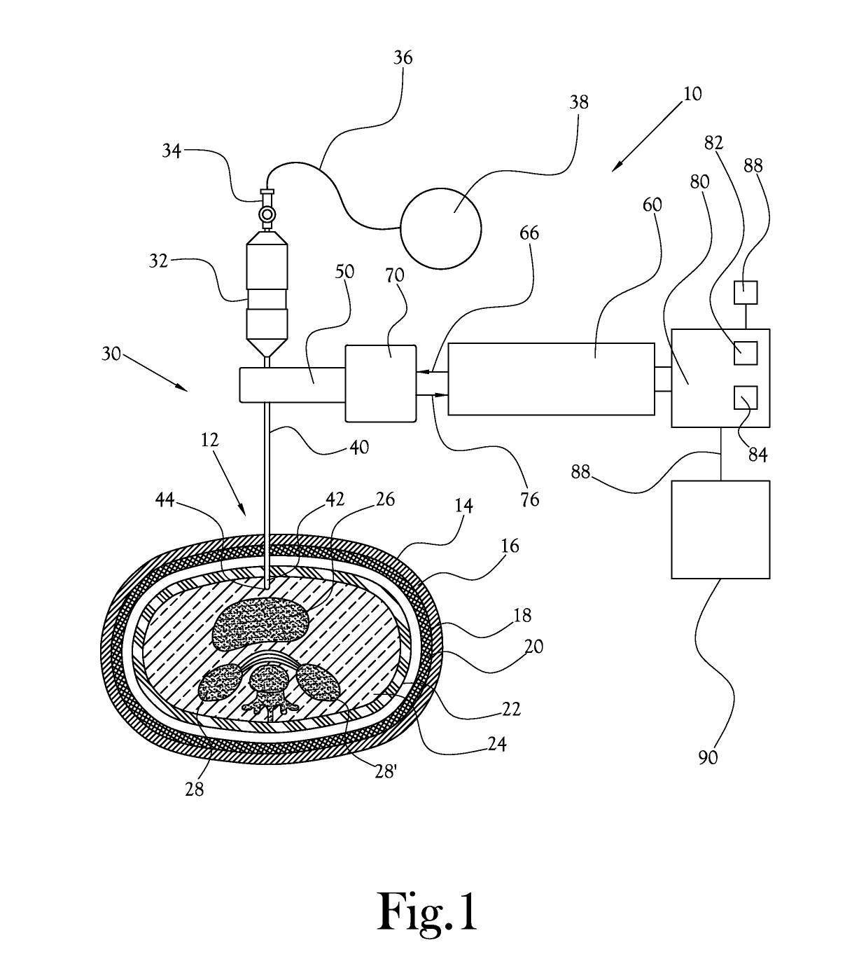

FIG. 1 illustrates a medical instrument penetration detector system according to an example embodiment of the present general inventive concept. The example medical instrument penetration detector system 10 illustrated in FIG. 1 includes a skin and tissue probe device, in this example a verres needle unit 30 that is utilized for surgical examination of a body cavity 24 of a patient, serving as a conductive signal path. However, as previously discussed, the present general inventive concept is not limited to any particular type of medical instrument/procedure. Any of a variety of medical instruments, such as a probe, cannula, trocar, etc., could be utilized as the conductive signal path in the penetration detector system 10. In the various example embodiments described herein, the terms conductive instrument and conductive signal path may be used interchangeably. This example embodiment can be used for the insertion of a distal penetration end 42 of the verres needle unit 30 through the layers of tissue of an umbilical region 12 of the patient's abdomen. The layers of tissue covering the umbilical region 12 typically include an outer or first surface layer of skin 14, a second layer of fat cells 16, followed b a fascia layer 18 and a layer of muscle 20. The layers of fascia and muscle may vary significantly in thickness between patients. An inner layer includes a peritoneum 22 that forms the lining of the abdominal cavity 24.

Referring to FIG. 1, the verres needle unit 30 is positioned to be inserted through the respective layers of tissue of the patient, with the needle or probe length between the proximal end 40 and the distal penetration end 42 serving as the conductive signal path when coupled to a properly grounded electrical conductor 50. A signal generator 80 is coupled to the electrical conductor 50 to automatically generate and transmit a plurality of signals of selected frequencies to the antenna.

The signal generator 80 may be programmed to apply a plurality of signal frequencies to be transmitted to the conductive signal path, which in this example is the verres needle unit 30. For instance, in various example embodiments 10 different signal frequencies in a range of 5 MHz to 50 MHz may be transmitted to the conductive path, in ascending increments of 5 MHz each. However, it is understood that this is simply one example of the range of signal frequencies that may be applied, as well as one example of the increments between signal frequencies. Various other frequencies and increments between frequencies may be used. It is also not necessary that the signal frequencies be generated and transmitted in ascending increments. Moreover, as described in more detail below, in connection with FIG. 10, it is possible to generate the multiple signal frequencies simultaneously using a broadband signal. Each of the plurality of signal frequencies transmitted to the conductive signal path may be evaluated according to one or more feedback parameters, as discussed later in more detail, to determine which of the signal frequencies is the most sensitive to changes in bio-impedance, e.g., the signal frequency for which the evaluated one or more feedback parameters indicate more significant information relative to the other generated frequencies, for the particular patient upon which the medical procedure is to be performed. For example, the “most sensitive” signal frequency might be the one of the evaluated signal frequencies having the most significant (e.g., highest or lowest) voltage standing wave ratio (VSWR), impedance, reflected power, propagated power, admittance, reflectance, transmittance, absorbance, etc., when compared to the frequencies of the other propagated signals. Data pertaining to the evaluation of each of the respective signal frequencies may be stored, so that the signal frequency that is most sensitive for the current patient may be chosen from all of the stored evaluated signals, and that signal frequency may then be set to be used for the remainder of the medical procedure as the operational frequency, or at least until the desired body location has been detected using the most sensitive signal frequency. This most sensitive signal frequency may be referred to as the operational signal frequency, and the signal corresponding to the most sensitive signal frequency as the operational signal. As previously discussed, due to various factors such as different body sizes, densities, bio-impedance, etc., a signal frequency of, for example, 25 MHz may be the most sensitive regarding various feedback parameters for one patient, while a signal frequency of, for example, 40 MHz may be the most sensitive for another patient. Throughout these descriptions, the most sensitive signal frequency refers to the most sensitive of the evaluated signal frequencies generated by the signal generator 80. For example, the most sensitive signal frequency may be the signal frequency that couples most efficiently with the particular patient upon which the medical procedure is to be performed. It is noted that the term “most sensitive” is used for convenience of description, and the term is not limited to any particular value or selection criteria.

In various example embodiments, the plurality of signal frequencies may be successively applied to the verres needle unit 30, so that the one or more feedback parameters of the respectively applied signals may be evaluated. In the illustrated example, the signal generator 80 includes a programmable microcontroller 82 to control the generation of the plurality of signal frequencies transmitted to the conductive signal path. However, in other various example embodiments the signal generator 80 may be controlled to generate the plurality of signal frequencies by a separate controller in electrical communication with the signal generator 80, such as a standalone computer or other such self-contained digital device. Similarly, though the signal generator 80 in the example embodiment illustrated in FIG. 1 includes a memory 84 to store various data such as a control program, evaluation results, etc., other various example embodiments may include memories provided in electrical communication with the signal generator 80, rather than located in the same unit. For example, the memory 84 may be stored in a separate module along with the microcontroller 82, or may be stored separately from both the microcontroller 82 and the signal generator 80. In various example embodiments, the signal generator 80 may be integrated with the medical instrument or may be formed as a separate unit.

Upon the determination of the most sensitive signal frequency, the microcontroller 82 may cause an indicator 86 to notify the user that the most sensitive signal frequency has been determined and set as the signal frequency that will be generated and transmitted for the remainder of the medical procedure. The indicator 86 may provide this notification audibly or visibly, or by an audio/visual combination. For example, the indicator 86 may be provided with a relatively simple circuit to provide an audible tone, a light indicator such as an LED, or other similar types of indicators, or any combination of such indicators, which indicate that the process of determining the most sensitive signal frequency, from the plurality of generated and evaluated signal frequencies, has been determined and set for the medical procedure. For example, a change in tone, a red light/green light configuration, or other such go/no-go type signal may be used to signal to the operator that a calibration of the operational frequency has been completed, and/or to indicate that a targeted body location has been reached by the medical instrument.

As illustrated in FIG. 1, the verres needle unit 30 may include various associated equipment known to those skilled in the art, such as a housing 32, a valve 34, a fluid or gas feeder line 36, and a fluid or gas storage reservoir 38. The needle includes a proximal end 40 that may be coupled to an electrical connector arm 50, and a distal penetration end 42. The penetration end 42 may include a fluid flow passage therein, such as a dispensing hole 44 for dispensing a fluid during a medical procedure as the penetration end penetrates body regions and/or when the penetration end reaches a targeted body region of the patient. As discussed in more detail later in this description, one or more various feedback parameters may be evaluated in the determination of the most sensitive signal frequency, such as, but not limited to, impedance, admittance, reflectance, transmittance, absorbance, standing wave ratio, etc., of the needle unit 30.

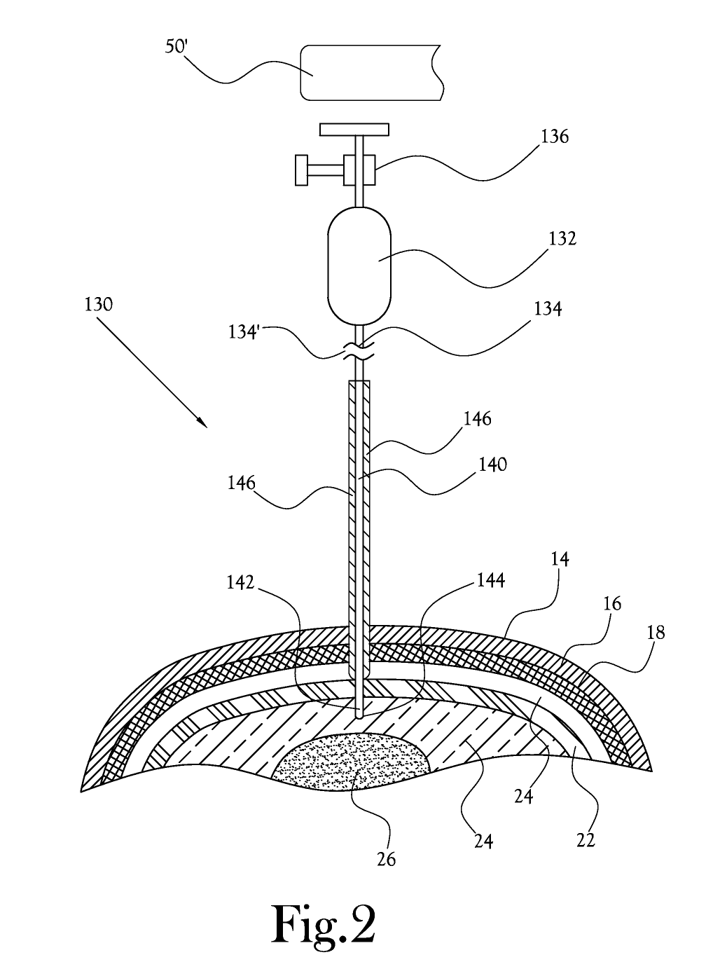

FIG. 2 illustrates a medical instrument penetration detector system according to another example embodiment of the present general inventive concept. The system illustrated in FIG. 2 includes a needle 130 similar to that of the verres needle unit 30 of FIG. 1, but having a selected base length portion 140 enclosed in an insulating layer 146. The needle 130 includes a handle portion 132, a junction 134, and a valve 136 at a manipulation end of the needle 130. The junction 134 includes a direct electrical connection of the base length portion 140 to the handle portion 132, for capacitive coupling with an electrical connector arm 50′. In other various example embodiments, the junction 134 may include a gap junction 134′ to allow inductive coupling across the gap junction 134′ to couple the base length portion 140 with an electromagnetic coil (not shown) to transmit input signals to the base length portion 140, or to receive feedback parameter signals reflected from the base length portion 140. An uninsulated needle insertion end 142 provides optimal electrical coupling with each respective layer of tissue 14, 16, 18, 20, 22 through which the insertion end 142 is inserted. The uninsulated insertion end 142 provides a probe that may more precisely monitor the feedback values of each respective body location that is contacted, since only the tip of the needle is in contact with the various anatomical regions of the body as the needle penetrates the body. In other words, as the uninsulated insertion end 142 of the conductive instrument, the needle 130 having an insulated base length 140 is inserted through the body (for example, through various anatomical regions and/or layers of tissue of the body), the feedback values detected at the insertion end 142 can be isolated from interference created by other layers of tissue above the insertion end 142. The insertion end 142 may include a dispensing hole 144 therein. An alternative insertion end for a verres needle may be retractable (not shown).

Referring again to FIG. 1, the signal generator 80 may include circuitry and a connection 88 to a power source 90 to provide electrical power to generate the plurality of signals at the plurality of frequencies. According to various example embodiments, the power source 90 may be an AC source supplied from outside the system 10, a DC battery source provided from within the system, and so on. The signal generator 80 may include circuitry to transmit the plurality of signals to the conductive signal path, which in this example is the verres needle unit 30. In various example embodiments, the microcontroller 82 controls the signal generator to adjust the range of the magnitude and frequency of the plurality of signals which will be automatically cycled through the defined spectrum to determine the most sensitive signal frequency, or waveform within the spectrum, for the particular patient upon which the medical procedure is to be performed. For example, the range of signal frequencies may be between approximately 100 kHz and approximately 1 GHz. However, it is understood that this is merely one example of the range of signal frequencies from which the plurality of signal frequencies may be chosen and tested. For instance, in various example embodiments, the microcontroller 82 may control the signal generator to generate ten different signal frequencies to be evaluated, the first signal frequency being 10 MHz, and the next 9 signal frequencies being raised in increments of 5 MHz each. However, it is understood that the number of signal frequencies that will be tested, as well as the respective values of the respective signal frequencies, are not limited to such an example. Before initiating the generation and transmission of the plurality of signal frequencies, the medical instrument, in this case the verres needle unit 30, may be inserted a predetermined distance into the body of the patient. In various example embodiments, the medical instrument may be inserted approximately 1 cm into the body before beginning the determination of the most sensitive signal frequency, and may then be inserted further, retracted, removed from the body, etc., before continuing with the medical procedure using the determined most sensitive signal frequency.

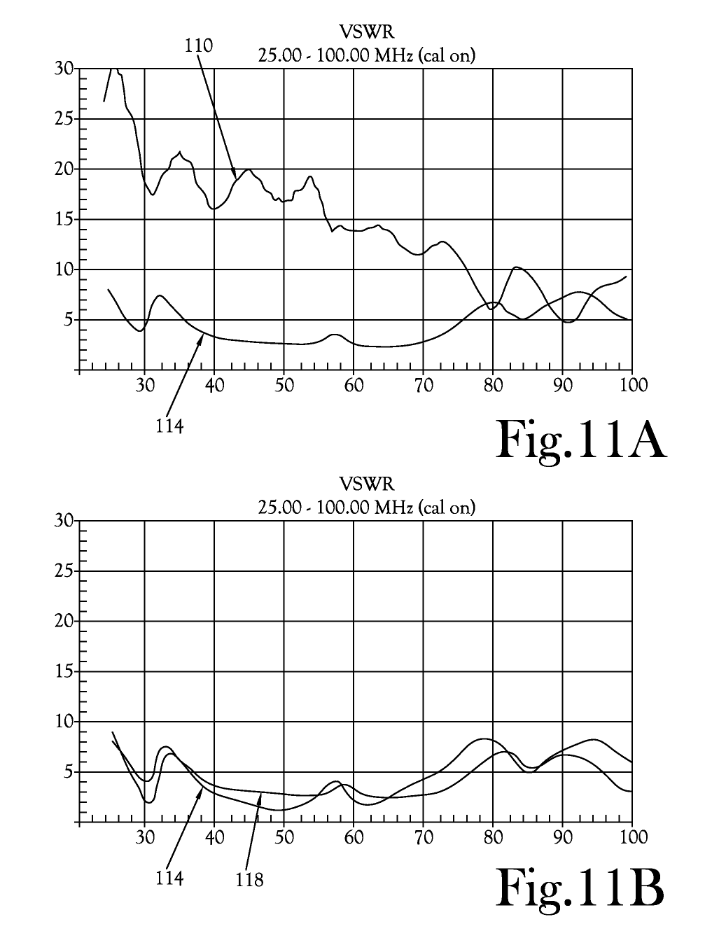

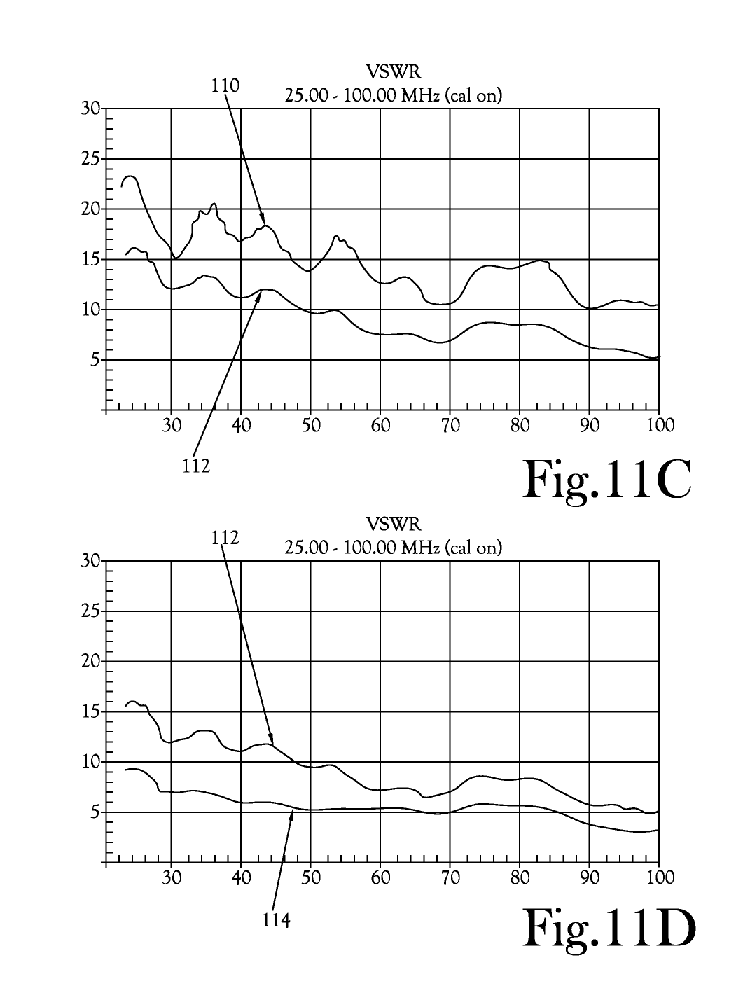

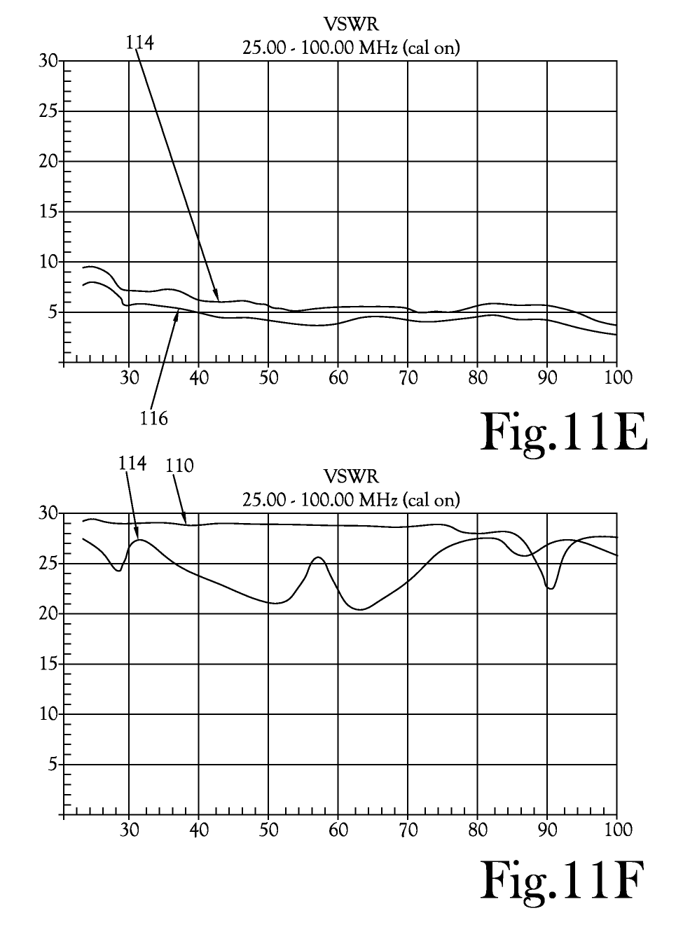

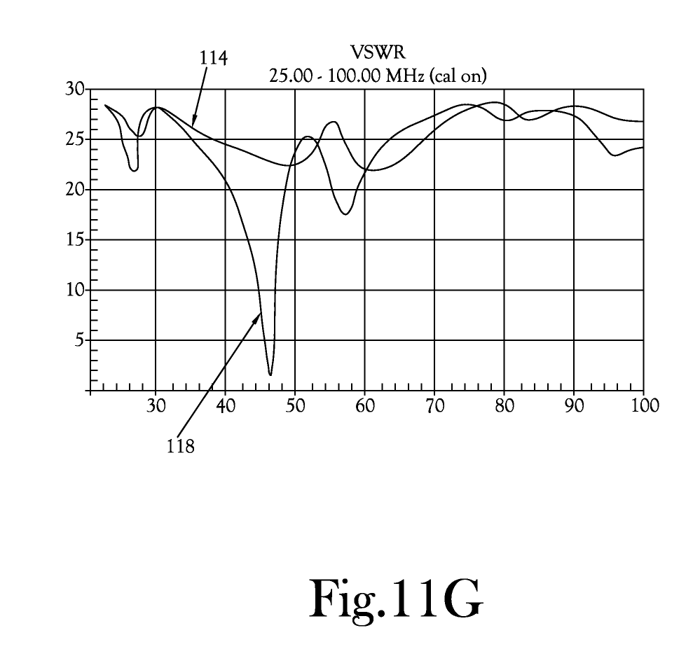

The medical instrument penetration detector system 10 of this example includes a detector 60 having circuitry to measure changes in one or more selected feedback parameters from the medical instrument serving as the conductive signal path, which in FIG. 1 includes the verres needle unit 30. The selected feedback parameters may include any one or a combination of the following parameters: VSWR, angle of reflective coefficient, reactance, impedance, phase shift coefficient, return power loss, reflected power, transmitted power, reflection coefficient, resistance, capacitance, inductance, admittance, reflectance, transmittance, absorbance, transmission loss, time domain reflectometry, and/or additional parameters related to signal frequency propagation as known to those skilled in the art. In various example embodiments, a detector 60 includes circuitry that measures the appropriately selected one or more feedback parameters, and may include audio and/or visual display notification equipment that issues alert signals and/or displays the VSWR, reactance, and/or any of the parameters identified above, or other parameters related to signal propagation and transmission line performance as known to those skilled in the art. The display notification equipment may include a visual display such as a display monitor or graphing equipment to display the selected feedback parameter, as illustrated in the graphs of FIGS. 11a-11g (which will be described later). In various example embodiments, the display could be an array of one or more Light Emitting Diodes (LEDs) or other light sources to give much simpler indications, such as a simple go/no-go indication. In other various example embodiments, a visual display may not be included, or an audio signal may be used.

It has been determined that the detected values for the one or more evaluated feedback parameters can be influenced by the selected length between the proximal end 40 and the penetrating end 42. The length of the instrument can be selected by an operator in various example embodiments to maximize the transmission/detection of the one or more feedback parameters. The selected length may be determined and used for the determination of the most sensitive signal frequency before commencing the actual medical procedure. After determining the most sensitive signal frequency and setting that signal frequency to be used in the medical procedure, the detected values for the one or more feedback parameters are further influenced by the movement of the probe serving as the medical instrument as the probe penetrates and contacts different body regions as the probe reaches the targeted body location of a patient.

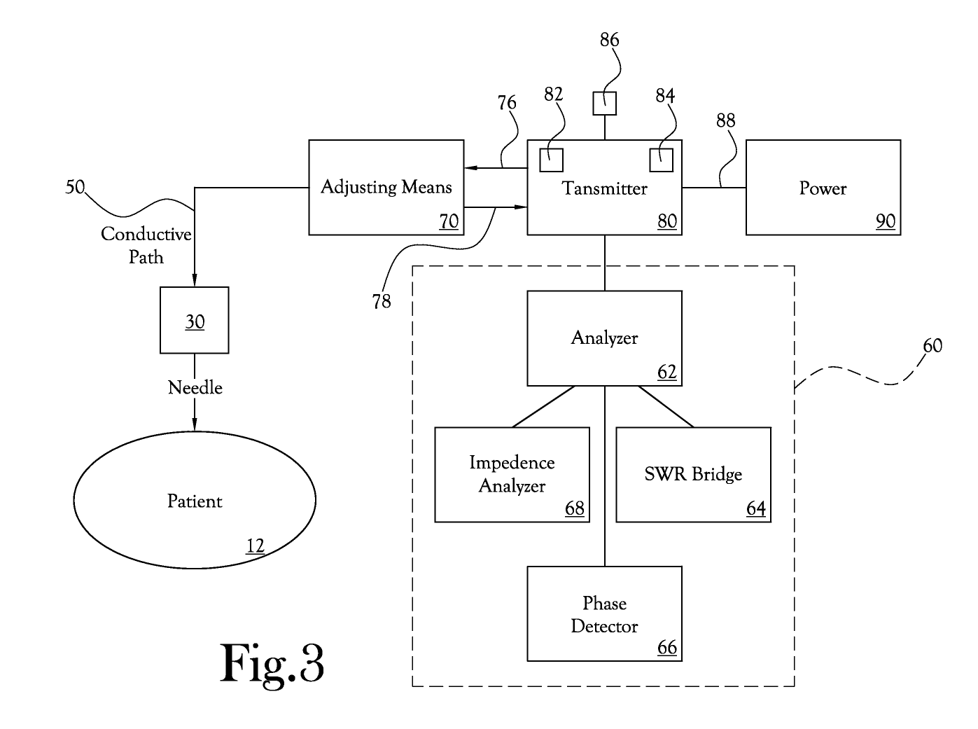

FIG. 3 illustrates a partial schematic view of various elements of the system illustrated in FIG. 1. In this example embodiment, the detector 60 includes an analyzer 62, including associated circuitry and controls for analysis and computation of a standard wave ratio (SWR) by an SWR bridge 64 to compare the magnitude of the one or more feedback parameters 78 seen by the needle unit 30 and/or measured by another feedback sensor. In various example embodiments, the detector 60 may further include an impedance analyzer 68 and circuitry for the measurement of the complex impedance (z) in ohms of the needle unit 30 that serves as the conductive signal path. The measurement circuitry may include a complex impedance analyzer 68 known to those skilled in the art. A typical unit that provides measurements of the VSWR, plus measuring and monitoring the return loss, is an Anritsu Wiltron 331A, or comparable models that are commercially available. Other specialized instruments are available to measure various feedback parameters such as the angle of reflective coefficient, reactance, complex impedance, phase shift coefficient, return power loss, reflected power, reflection/transmission coefficient, true resistance, capacitance, inductance, transmission loss, time domain reflectometry, absorbance, and/or additional parameters related to frequency signal transmissions as known to those skilled in the art, and may be included according to various example embodiments of the present general inventive concept. The detector 60 may be used to evaluate the one or more feedback parameters to determine the most sensitive signal frequency, and to evaluate the one or more feedback parameters during the ensuing medical procedure to determine location of the medical instrument in the body using the selected frequency.

The detector 60 may also include a phase detector 66 to detect phase shifting of the feedback parameters 78. In various example embodiments, the analyzer and circuitry 62 utilizes the SWR bridge 64 to compare the wave characteristics of the selected feedback parameters 78. The resulting change of the one or more feedback parameters may be calculated for each of the respective generated signal frequencies to determine and select of the most sensitive signal frequency, and, after the determination of that most sensitive signal frequency, to measure changes in the one or more feedback parameters as the probe travels through each respective body location, such as tissue layers, body cavities, etc., of the patient. The detector 60 and analyzer 62 may include circuitry and a feedback notification device such as a visual and/or an audible indicator that indicates by an alert signal to an operator when each respective body location it penetrated. Further, according to various example embodiments, the detector 60 and analyzer 62 may have a visual and/or audible indicator to indicate that the most sensitive signal frequency has been determined. In other various example embodiments, the visual and/or audible indicators to indicate that the most sensitive signal frequency has been determined may be provided to the signal generator 80, a separate module, and so on.

A grounded electrical connection 50 may be maintained between the needle unit 30 and the signal generator 80, and the detector 60 and analyzer 62. The detector 60 and analyzer 62 may further include analysis circuitry to compare signal changes as the plurality of different signal frequencies are generated, and, after the determination and setting of the most sensitive signal frequency, as the penetrating end 42 is manipulated by the depth adjusting element 70.

When determining the most sensitive signal frequency, an operator may insert a tip of the medical instrument, in this example the distal end 42 of the needle unit 30, approximately one centimeter into the body of the patient. A calibration process may then be initiated by the operator pressing a button, switch, etc., at which point the microcontroller 82 may control the signal generator 80 to generate and transmit the first of the plurality of signal frequencies, and may store the results of the detector 60 and analyzer 62 corresponding to that first signal frequency in the memory 84. After storage of the analysis results corresponding to the first signal frequency in the memory 84, the microcontroller 82 may control the signal generator 80 to generate and transmit the second of the plurality of signal frequencies, and may store the corresponding analysis results in the memory 84. This process may be repeated for each of the prescribed number of generated signal frequencies, and after the prescribed number of generated signal frequencies have been generated and the respective corresponding results have been stored in the memory 84, the results may be compared with one another to determine the most sensitive signal frequency. In other words, of all of the plurality of the generated signal frequencies, the one signal frequency having corresponding results which show a desired value in the one or more feedback parameters for a particular patient can be determined to be the most sensitive signal frequency, and that signal frequency will be selected and used as the operational signal during the medical procedure.

After the selected signal frequency has been determined and set for the medical procedure, the complex impedance for the needle unit 30 may be analyzed during passage through each tissue layer. As an example to illustrate the different type of values that may be encountered, a hypothetical case will be considered in which a selected signal frequency for a current patient has been determined to be 55 MHz, and that signal frequency has been set to be generated for the remainder of the medical procedure. If an operator were to remove the needle unit 30 from the patient after the setting of the desired frequency (55 MHz in this hypothetical example), the operator may observe that the VSWR of the needle unit 30 in air, i.e., not inserted into or against the patient's tissue, may be about 15 to about 22. As the needle unit 30 is placed on the outer skin layer 14, a VSWR may be observed of about 7 to about 10. As the penetrating end 42 is inserted through the skin layer 14, a VSWR may be observed of about 5 to about 6. When the penetrating end 42 is inserted into the peritoneum 22, a VSWR may be observed of about 1 to about 4, which allows the operator to confirm that the penetrating end 42 has reached the targeted body location.

In the confines of an operating room, an assistant may be requested to perform the following operations to provide the medical instrument penetration detector system 10 and to confirm that a needle penetrating end 42 is properly inserted into a selected body location such as an abdominal cavity 24. Power may be provided by a shielded power line 88 from a power source 90. The signal input 76, being the selected operational signal frequency, may be transmitted by a conductive path including the depth adjusting element 70, the grounded electrical connection 50, to the needle unit 30 serving as the conductive instrument. As the depth of insertion of the penetrating end 42 is adjusted by the assistant with the depth adjusting element 70, the one or more feedback parameters 78 may be transmitted from the needle unit 30 to the analyzer circuitry 62 for computation. Using the values of the example discussed above with the hypothetical patient for whom the selected signal frequency was 55 MHz, when the VSWR of the needle unit 30 approaches about 1 to about 4, the assistant may confirm that the penetrating end 42 of the needle (or other medical instrument) is properly inserted through the body tissue and into the body location selected for investigation, such as the abdominal cavity 24.

The medical instrument penetration detector system 10 may be utilized to confirm proper insertion of the penetrating end 42 into the abdominal cavity 24 as illustrated above, or for any number of other medical procedures, such as insertion of spinal or epidermal catheters into the layers below or above the spinal membrane, to confirm proper insertion of a subclavian catheter in to the subclavian vein, for placement of a needle, probe, cannula, troca, catheter, etc., into the pleural cavity of the chest, bladder, joint spaces, extremity veins or arteries, or any body location, such as a body cavity or tissue space, of the patient, and so on.

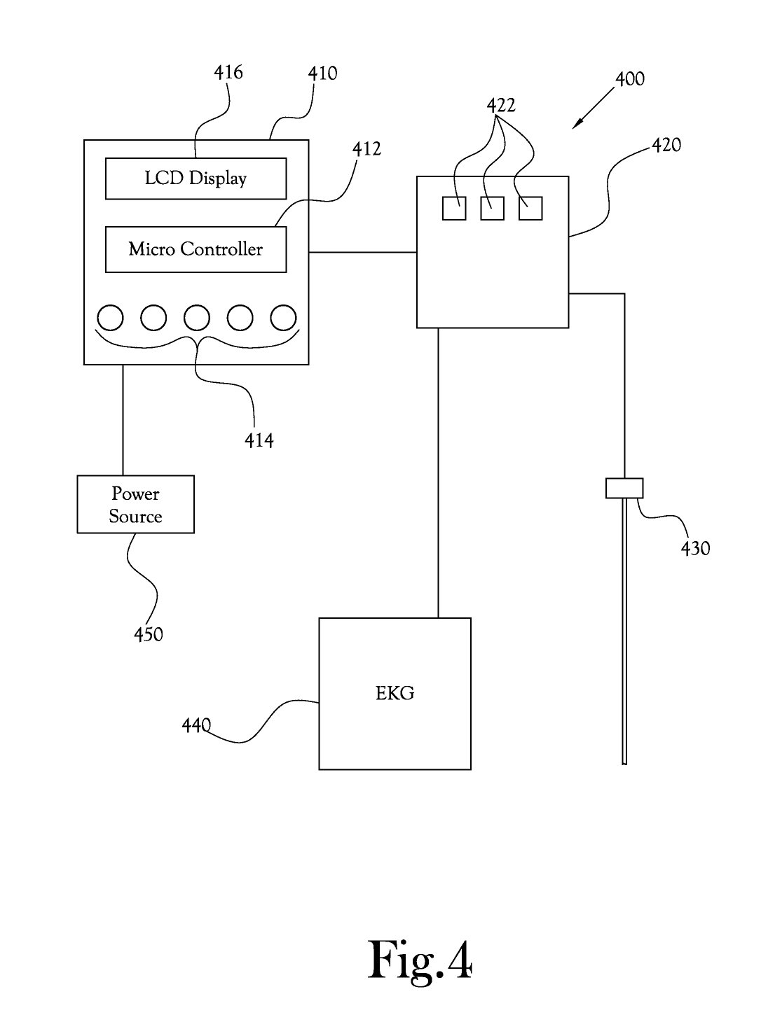

FIG. 4 illustrates a medical instrument penetration detector system according to another example embodiment of the present general inventive concept. The example system 400 of FIG. 4 includes a digital module 410 in electrical connection with an analog module 420 which is also in electrical communication with a conductive instrument, such as a probe 430, and an EKG pad 440 which is to be affixed to a patient to provide a reference voltage. The digital module 410 may include a controller to control operations of the system 400, and controls the analog module 420 to generate an incident analog signal at a plurality of signal frequencies to be transmitted to the probe 430. The analog module may include various circuitry 422 for the detection and analysis of various feedback parameters corresponding to the respective generated signal frequencies.

In the illustrated embodiment, the digital module 410 includes a microcontroller 412 to control various operations of the digital module 410, as well as the overall system 400. The digital module is in electrical communication with a power source 450, which may be a battery, an AC source, and so on. In other various embodiments, the power source 450 may be integrated directly in the digital module 410. The digital module 410 may include a plurality of control buttons 414 which may control such functions as power on/off, the initiation of the calibration process, switching between display modes, etc. An LCD display 416 may be provided to display various modes in which the system 400 is operating, analysis results, and so on.

The probe 430 and EKG pad 440, as well as the wires providing the electrical communication to the analog module 420, may be disposable, and may be connected to the analog module 420 by any of various physical connections.

The probe 430 may be inserted a predetermined distance, such as, for example, one centimeter, into the body tissue of the patient at an area proximate to the body location which is to be probed. After such insertion, an operator may press a calibration button (included in the control buttons 414) on the digital module 410, and the microcontroller 412 may control the analog module 420 to generate the first of a plurality of signal frequencies which are to be analyzed. The signal of this first frequency is transmitted to the probe 430, and one or more feedback parameters are analyzed by the analog module 420, the results of which may be stored in a memory provided to the digital module 410. After generating and evaluating a plurality of signal frequencies, the corresponding results stored in the memory are evaluated, and the signal frequency for which the corresponding results indicate a desired value for the feedback is selected as the operational frequency for that patient. In various example embodiments, the selected signal frequency is displayed on the LCD display 416. In other various example embodiments, the LCD display 416 may simply indicate that the selected signal frequency has been determined and set as the operational signal frequency to be generated for the remainder of the medical procedure. In even other various example embodiments, an audible indicator, such as a tone, may indicate that the desired operational signal frequency has been determined. Some example embodiments may combine the visual and audible indicators.

After the user has been notified that the operational signal frequency has been selected, the digital module 410 controls the analog module 420 to generate the signal at the selected signal frequency, and the operator may continue the medical procedure using the selected frequency. It is understood that the while a probe 430 is described as an example medical instrument in this example, any number of medical instruments may serve as the conductive instrument, according to the instruments desired for different respective medical procedures.

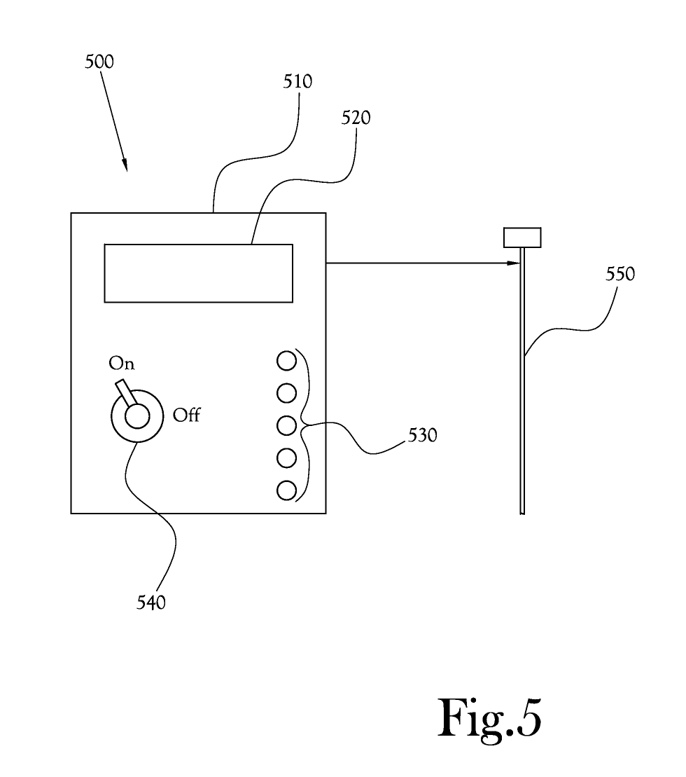

FIG. 5 illustrates a medical instrument penetration detector system according to yet another example embodiment of the present general inventive concept. The system 500 is similar to the system 400 illustrated in FIG. 4, but includes a control module 510 which combines the digital module 410 and analog module 420 illustrated in FIG. 4. The control module 510 includes an LCD display 520 to display generated signal frequencies, feedback parameter evaluation results, etc., control buttons 530 which may control such functions as the initiation of the calibration process, switching between display modes of different feedback parameters, etc., and an on/off switch 540. The system 500 may perform substantially similar functions, in a substantially similar fashion, as the system 400 illustrated in FIG. 4, with the convenience of a relatively portable, e.g., handheld, control module provided with control circuitry as well as signal generation and evaluation frequency contained therein. A medical instrument 550 serving as the conductive instrument may be connected directly to the control module 510.

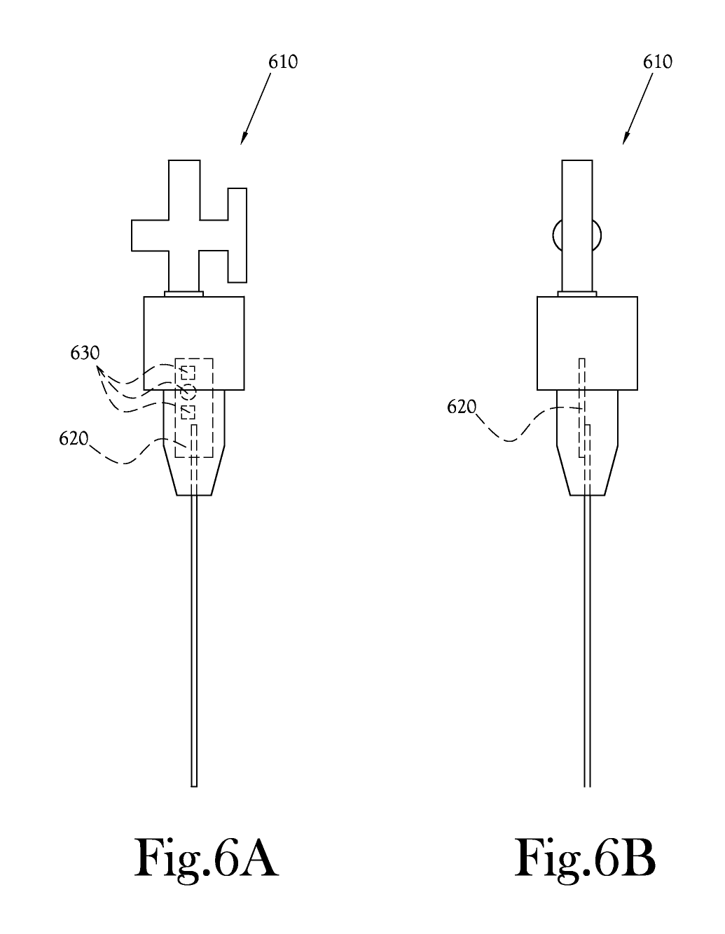

FIGS. 6A-6B illustrate a front and side view of an example medical instrument provided with signal generating electronics according to an example embodiment of the present general inventive concept. The medical instrument 610 may be one of various conductive medical instruments such as, for example, a probe, trocar, cannula, needle, etc. Control circuitry, such as 620, can be provided to the medical instrument 610. The signal transmitted along the conductive path of the conductive medical instrument 610 may be generated and evaluated by various signal generation and evaluation circuitry 630 integrated with the electronics board 620 provided to the medical instrument 610. In various example embodiments, the electronics board 620 may be provided in a readily detachable fashion to the medical instrument 610. In other words, according to various example embodiments, the electronics board 620 may be plugged into a corresponding socket provided in the medical instrument 610, slid into a corresponding slot, and so on. In other various example embodiments, the electronics board 620 may be provided as an integrated portion of the medical instrument 610. The electronics board 620 may be controlled by digital control circuitry to generate a plurality of signal frequencies to be transmitted to the conductive medical instrument 610, the signal frequencies being evaluated to select the desired operational frequency according to one or more evaluated feedback parameters. The electronics board 620 may be provided with a microcontroller and memory in the circuitry 630, or in other various example embodiments the electronics board 630 may be in electrical communication with a separately provided controller and memory. The medical instrument 610 may also be provided with manual controls such as a calibration initiation button 640 to be used by an operator to initiate the process of determining the most sensitive signal frequency, and an indicator 650 to indicate to the use that the selected frequency has been determined and set to be used for the remainder of the medical procedure. The indicator 650 may be a visual indicator, such as a light emitter, an audible indicator, such as a simple speaker emitting a single tone, or a combination visual/audible indicator. In various example embodiments, the indicator 650 may be a combination of two lights, in which a red light is on during the signal frequency calibration process, and a green light is turned on, along with the red light being turned off, to indicate that the calibration process has been completed.

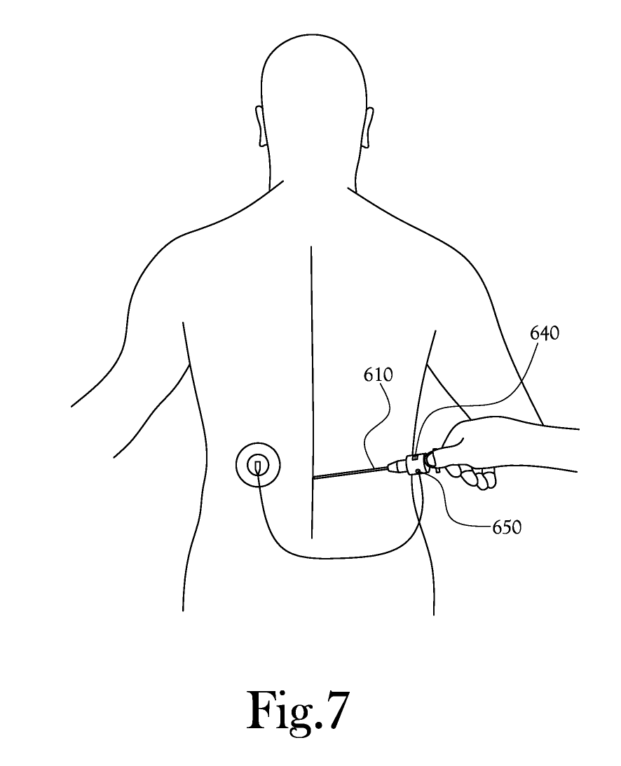

FIG. 7 illustrates a medical procedure using the conductive medical instrument illustrated in FIG. 6. In this simplified illustration, a user is inserting a portion of the medical instrument 610 into the body of a patient. As an example of the calibration operation for the medical instrument 610, the user may insert the distal end of the medical instrument 610 approximately one centimeter into the body tissue of the patient, and press the calibration initiation button 640. The signal generation and evaluation circuitry 630 of the electronics board 620 may then generate a plurality of incident waveforms having different frequencies, and evaluate one or more feedback parameters based on characteristics of the patient's body to determine the most effective signal frequency of the evaluated signal frequencies. Upon determining the most effective signal frequency, that signal frequency is set to be generated for the remainder of the medical procedure, and the indicator 650 controlled to indicate to the operator that the operational frequency has been set.

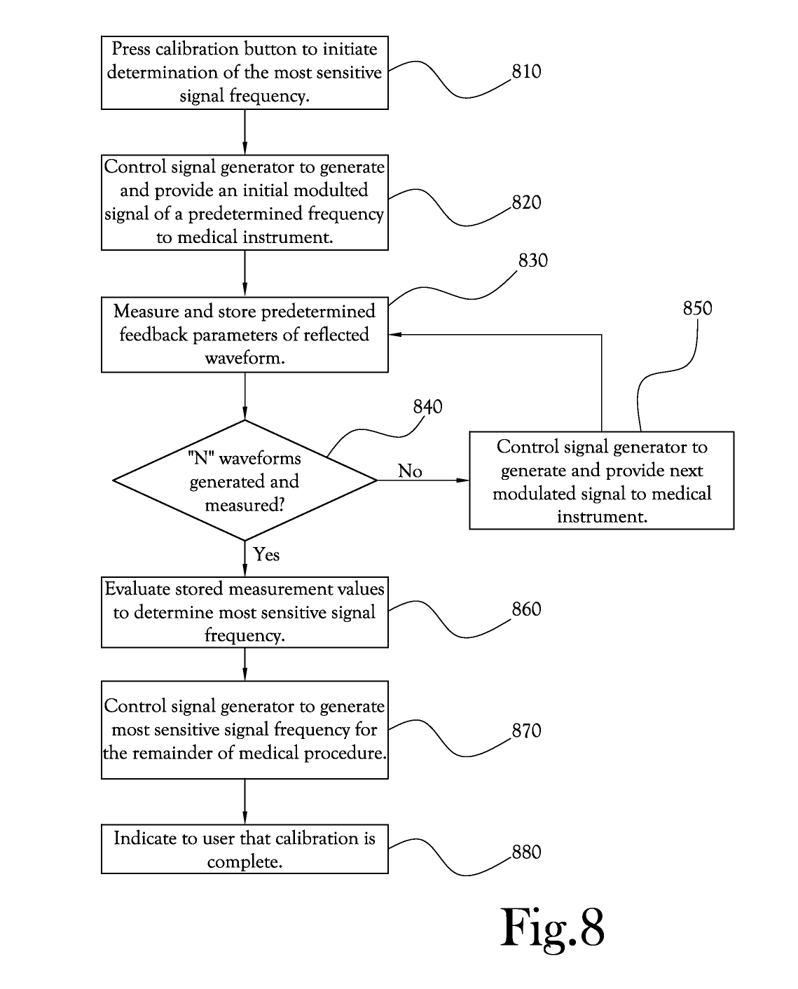

FIG. 8 illustrates a flow chart of a method of dynamic frequency adjustment of a conductive medical instrument according to an example embodiment of the present general inventive concept. In operation 810, a user initiates the determination of the operational signal frequency by pressing a calibration button. In operation 820, a signal generator is controlled to generate a first signal of a predetermined frequency, and the generated signal is provided to the conductive signal path of the conductive medical instrument. In operation 830, one or more predetermined feedback parameters are measured and stored. In operation 840, it is determined whether a predetermined number n of waveforms have been generated and evaluated. For example, if the predetermined number of waveforms to be generated and evaluated is 10, it is determined whether ten waveforms have been generated and evaluated. However, it is understood that various other quantities of waveforms, other than 10, may also be used. If the predetermined number n of waveforms have not been generated, in operation 850 the signal generator is controlled to generate another signal having a different predetermined frequency than the previously generated signal. For example, the signal generator may be controlled to increment the frequency so that the newly generated signal has a frequency that is 5 MHz higher than the previously generated signal. After the newly generated signal has been provided to the conductive medical instrument, operation 830 is repeated to measure and store the one or more predetermined feedback parameters corresponding to the newly generated signal.

If it is determined in operation 840 that the predetermined number N of frequencies have been generated and evaluated, in operation 860 the measurement values stored in operation 830 are evaluated to determine which corresponding frequency generated the most significant feedback parameter measurement. In operation 870 the signal generator is controlled to generate the signal at the selected frequency determined in operation 860 for the remainder of the medical procedure. In operation 880 an audio and/or visual indicator is controlled to indicate to the user that the calibration is complete. In other words, the user is informed by the indicator that the signal generator is generating and providing the selected signal frequency to the conductive medical device, and the user may now proceed with the medical procedure using an optimum frequency for that particular patient.

In various example embodiments, software and/or firmware controlling various operations of the components may consider particular patient characteristics such as bio-impedance differences due to age, gender, Body Mass Index (BMI), physical condition, and the like to determine which feedback parameters to measure and evaluate. A stored look-up table may be incorporated in some example embodiments to choose the feedback parameters based on such patient characteristics. In other various example embodiments, the user may choose the one or more feedback parameters used to evaluate the propagated signal.

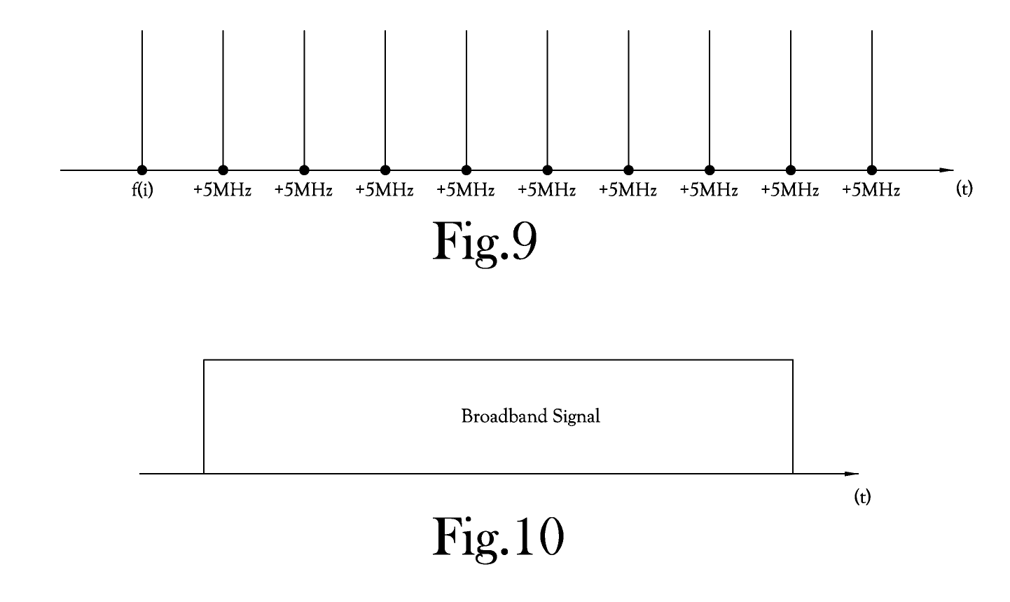

FIG. 9 illustrates a timeline over which a plurality of generated signal frequencies are increasingly incremented according to an example embodiment of the present general inventive concept. As previously described, a signal generator may be controlled to generate a signal at an initial frequency f(i), and then increasingly increment the signal frequency after one or more various feedback parameters corresponding to each of the generated frequencies are measured and the results are stored. In the example illustrated in FIG. 9, the signal generator is controlled to increase the generated signal frequency by 5 MHz at each increment. Thus, in one example embodiment, the initial generated signal frequency f(i) may be 15 MHz, and the signal frequency may be increased in increments of 5 MHz until the last signal frequency of 60 MHz is generated and evaluated. After the one or more stored feedback parameters from the generated signals are evaluated to determine which of the corresponding signal frequencies demonstrates a desired feedback value, the signal frequency is set to such frequency for the remainder of the medical procedure. It is understood that the frequencies, quantity of generated signal frequencies, and increment values described in this described embodiment are merely examples, and any of these values may change according to various example embodiments of the present general inventive concept. Also, the various signal frequencies may be continuously transmitted until the signal frequency is changed, or there may be intermittent pauses in the generation of the plurality of signal frequencies. For example, an initial signal frequency f(i) may be 80 MHz, the signal frequencies may be decreased over time, the signal frequencies may be changed in increments of 10 MHz, more or fewer than ten signal frequencies may be evaluated, and so on.

FIG. 10 illustrates a timeline over which a plurality of generated signal frequencies are simultaneously generated according to an example embodiment of the present general inventive concept. As illustrated in FIG. 10, the signal generator may be configured to generate a broadband signal output in which the plurality of generated signal frequencies are simultaneously generated. In various example embodiments in which a broadband output signal such as this is generated, the receiver circuitry of the detector may be configured as a narrowband receiver which incrementally detects specific channels within the overall range of frequencies being generated in the broadband signal, and may measure and store one or more predetermined feedback parameters associated with the respective frequencies corresponding to those channels. In such example embodiments, the operational signal frequency may be determined in a similar fashion as the previously described example embodiments. In other example embodiments, a broadband signal may be generated, and broadband receiver circuitry may be used to detect an overall change in the properties, such as total energy, of the broadband signals as different body locations are encountered by a conductive medical instrument in a medical procedure.

FIGS. 11A-11G are graphs illustrating changes in a feedback parameter as a medical instrument is moved to various body locations. In these graphs, it is assumed that an optimal operational signal frequency has already been determined and set for the medical procedure. The y-axis of FIGS. 11a-11e, entitled VSWR for voltage standing wave ratio, is a unit-less value for standing wave voltage ratio. The y-axis of FIGS. 11f-11g, entitled Return Loss, is in decibel (db). The x-axis of FIGS. 11a-11g is a wavelength value measured in megahertz.

It is assumed for the purpose of this example that the medical instrument to which the optimal operational signal frequency is being applied has a characteristic impedance value 111 while suspended in air (see FIGS. 11a, 11c, and 11f), with a different impedance 112 obtained when the instrument is placed on a patient's skin (see FIGS. 11c and 11d), or placed below the patient's skin (see FIGS. 11a, 11b, 11d). Therefore, there is a marked effect on the feedback parameter according to the body location in which the instrument is located. In this example embodiment, each of the body locations may cause a different impedance for the conductive medical instrument for which the determined frequency signal is generated. Therefore, there are different impedance values when the instrument is positioned on the skin (see FIGS. 11c and 11d), compared to when the distal end of the instrument has entered through the skin (see FIGS. 11a, 11b, 11d, 11e, 11f, and 11g), or has been placed deeper into respective layers of the tissue of the patient. In addition, the instrument will register a lower impedance 118 during penetration into the peritoneum (see FIGS. 11b and 11g), as compared to an impedance 116 during insertion into a small cavity such as a vein (see FIG. 11e). Therefore, when the medical practitioner seeks confirmation that the instrument is positioned in the targeted body location, for verification to proceed with a surgical procedure such as laparoscopic surgery, the practitioner simply need confirm that the impedance has reached the impedance matching value of a preselected value.

According to various embodiments of the present general inventive concept, a location of a medical instrument in a patient during a medical procedure can be determined connecting at least a portion of the medical instrument to a first body region of the patient, propagating a plurality of signals at different frequencies along a conductive path of the medical instrument, measuring one or more feedback parameters corresponding to each of the plurality of signals at the first body region, determining an operational frequency from the different frequencies according to a comparison of the one or more feedback parameters, propagating a signal having the operational frequency along the conductive path as the medical instrument penetrates the body of the patient during the medical procedure, and measuring the one or more feedback parameters corresponding to the operational frequency to determine the location of the medical instrument with respect to the body of the patient.

The concepts and techniques disclosed herein are not limited to any particular type of injected medical instrument, and could be applied to various other applications and objects, without departing from the scope and spirit of the present general inventive concept. For example, although the detection of a verres needle during a peritoneal procedure has been described and illustrated, any number of other procedures, such as epidural procedures, phlebotomy, and so on, which include the introduction of a medical instrument into the tissue of a patient may be performed according to various example embodiments of the present general inventive concept. Also, as previously described, the medical instruments used in these procedures may include any of a variety of medical instruments, such as a probe, trocar, cannula, needle, and so on.

It is noted that the simplified diagrams and drawings do not illustrate all the various connections and assemblies of the various components, however, those skilled in the art will understand how to implement such connections and assemblies, based on the illustrated components, figures, and descriptions provided herein, using sound engineering judgment.

The present general inventive concept can be embodied as computer- readable codes on a computer-readable medium. The computer-readable medium can include a computer-readable recording medium and a computer-readable transmission medium. The computer-readable recording medium is any data storage device that can store data as a program which can be thereafter read by a computer system. Examples of the computer-readable recording medium include read-only memory (ROM), random-access memory (RAM), CD-ROMs, DVDs, magnetic tapes, floppy disks, and optical data storage devices. The computer- readable recording medium can also be distributed over network coupled computer systems so that the computer-readable code is stored and executed in a distributed fashion. The computer-readable transmission medium can transmit carrier waves or signals (e.g., wired or wireless data transmission through the Internet). Also, functional programs, codes, and code segments to accomplish the present general inventive concept can be easily construed by programmers skilled in the art to which the present general inventive concept pertains.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

While the present general inventive concept has been illustrated by description of several example embodiments, it is not the intention of the applicant to restrict or in any way limit the scope of the inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings.

1. A method of determining a location of a medical instrument in a patient during a medical procedure, the method comprising:
connecting at least a portion of the medical instrument to a first body region of the patient;
propagating a plurality of signals at different frequencies along a conductive path of the medical instrument;
measuring one or more feedback parameters corresponding to each of the plurality of signals at the first body region;
determining an operational frequency from the different frequencies according to a comparison of the one or more feedback parameters;
propagating a signal having the operational frequency along the conductive path as the medical instrument penetrates the body of the patient during the medical procedure; and
measuring the one or more feedback parameters corresponding to the operational frequency to determine a penetration location of the medical instrument in the body of the patient.

2. The method of claim 1, further comprising generating at least one indicator to indicate the medical instrument has reached a targeted body region of the patient.

3. The method of claim 1, wherein the connecting includes injecting the medical instrument into the body of the patient.

4. The apparatus of claim 3, wherein the medical instrument is injected approximately one centimeter into the body of the patient.

5. The method of claim 1, wherein the feedback parameters include voltage standing wave ratio (VSWR), angle of reflective coefficient, reactance, impedance, phase shift coefficient, return power loss, reflected power, propagated power, reflection coefficient, resistance, capacitance, inductance, admittance, reflectance, absorbance, transmittance, transmission loss, time domain reflectometry, or any combination thereof.

6. The method of claim 1, further comprising storing the detected one or more feedback parameters with information associating the detected one or more feedback parameters with the respective corresponding frequencies.

7. The method of claim 1, wherein the medical instrument is a probe, trocar, cannula, or needle.

8. The method of claim 7, wherein the medical instrument is at least partially covered with an insulating material, having at least a portion of a distal end of the medical instrument connected to the patient exposed to contact the patient.

9. The method of claim 1, wherein the plurality of signals at different frequencies are propagated successively.

10. The method of claim 9, wherein the frequencies of the plurality of signals are incremented by a constant value.

11. The method of claim 10, wherein the frequencies of the plurality of signals are incremented by 3, 5, or 10 MHz.

12. The method of claim 9, wherein a quantity of 5, 7, 10, or 15 of the signals at different frequencies are propagated successively.

13. The method of claim 1, wherein the plurality of signals at different frequencies are propagated simultaneously in a broadband signal.

14. The method of claim 13, wherein the measuring includes incrementally adjusting band pass receiving circuitry to selectively receive channels corresponding to the plurality of signals at different frequencies in the broadband signal.

15. The method of claim 1, further comprising generating at least one indicator to indicate the signal having the operational frequency is being propagated along the conductive path.

16. The method of claim 15, wherein the at least one indicator includes at least one audible indicator, at least one visual indicator, or any combination thereof.

17. The method of claim 16, wherein at least one completion tone is emitted in response to the signal having the operational frequency being propagated along the conductive path.

18. The method of claim 17, wherein at least one processing tone is emitted in response to the determining of the operational frequency being in process.

19. The method of claim 15, wherein a completion visual indicator is turned on in response to the signal having the operational frequency being propagated along the conductive path.

20. The method of claim 19, wherein a processing visual indicator is turned on in response to the determining of the operation frequency being in process.

21. A system to determine a location of a medical instrument in a patient during a medical procedure, the system comprising:
a signal generator to propagate a plurality of signals at different frequencies along a conductive path of the medical instrument;
a measuring unit to measure one or more feedback parameters corresponding to each of the plurality of signals when the medical instrument is connected to a first body region of the patient;
a comparing unit to compare the one or more feedback parameters of the respective signals; and
a determining unit to determine an operational frequency from the different frequencies according to the comparison;
wherein the signal generator propagates a signal having the operational frequency along the conductive path as the medical instrument penetrates the body of the patient during the medical procedure; and
the measuring unit measures the one or more feedback parameters corresponding to the operational frequency to determine a penetration location of the medical instrument in the body of the patient.

22. The system of claim 21, further comprising at least one indicator to indicate the medical instrument has reached a targeted body region of the patient.

23. The system of claim 21, wherein the signal generator, measuring unit, comparing unit, and determining unit are provided to the body of the medical instrument.

24. The system of claim 21, wherein the feedback parameters include voltage standing wave ratio (VSWR), angle of reflective coefficient, reactance, impedance, phase shift coefficient, return power loss, reflected power, propagated power, reflection coefficient, resistance, capacitance, inductance, admittance, reflectance, absorbance, transmittance, transmission loss, time domain reflectometry, or any combination thereof.

25. The system of claim 21, further comprising a memory to store the detected one or more feedback parameters with information associating the detected one or more feedback parameters with the respective corresponding frequencies.

26. The system of claim 21, wherein the medical instrument is a probe, trocar, cannula, or needle.

27. The system of claim 26, wherein the medical instrument is at least partially covered with an insulating material, having at least a portion of a distal end of the medical instrument exposed to contact the patient.

28. The system of claim 21, wherein the signal generator successively propagates the plurality of signals at different frequencies.

29. The system of claim 28, wherein the signal generator increments the frequencies of the plurality of signals by a constant value.

30. The system of claim 29, wherein the signal generator increments the frequencies of the plurality of signals by 3, 5, or 10 MHz.

31. The system of claim 28, wherein the signal generator successively propagates a quantity of 5, 7, 10, or 15 of the signals at different frequencies.

32. The system of claim 25, wherein the signal generator propagates the plurality of signals at different frequencies simultaneously in a broadband signal.

33. The system of claim 32, wherein the measuring unit includes band pass receiving circuitry to selectively detect specific channels with an overall range of the broadband signal.

34. The system of claim 21, further comprising at least one indicator to indicate the propagation along the conductive path of the signal having the operational frequency.

35. The system of claim 34, wherein the at least one indicator includes at least one audible indicator, at least one visual indicator, or a combination thereof.

36. The system of claim 35, wherein the at least one audible indicator emits at least one completion tone in response to the propagation along the conductive path of the signal having the operational frequency.

37. The system of claim 36, wherein the at least one audible indicator emits at least one processing tone in response to the determining of the operational frequency being in process.

38. The system of claim 34, wherein the at least one indicator includes a first visual indicator that is turned on in response to the propagation along the conductive path of the signal having the operational frequency.

39. The system of claim 38, wherein the at least one indicator includes a second visual indicator that is turned on in response to the determining of the operational frequency being in process.

40. The system of claim 39, wherein the first visual indicator is green, and the second visual indicator is red.

41. A processor readable storage medium having recorded thereon a program to cause a processor to perform a method of determining a location of a medical instrument in a patient during a medical procedure, the method comprising:
connecting at least a portion of the medical instrument to a first body region of the patient;
propagating a plurality of signals at different frequencies along a conductive path of the medical instrument;
measuring one or more feedback parameters corresponding to each of the plurality of signals at the first body region;
determining an operational frequency from the different frequencies according to a comparison of the one or more feedback parameters;
propagating a signal having the operational frequency along the conductive path as the medical instrument penetrates the body of the patient during the medical procedure; and
measuring the one or more feedback parameters corresponding to the operational frequency to determine a penetration location of the medical instrument in the body of the patient.
